(19) United States
(12) Patent Application Publication
(10) Pub. No.: US 2016/0193051 A1
(43) Pub. Date: Jul. 7, 2016

(54) PATIENT-MATCHED ACETABULAR AUGMENT WITH ALIGNMENT GUIDE

(71) Applicant: SMITH & NEPHEW, INC., Memphis, TN (US)

(72) Inventors: Sachin P. Budhabhatti, Collierville, TN (US); Alisha W. Bergin, Southaven, MS (US)

(21) Appl. No.: 14/916,776

(22) PCT Filed: Sep. 5, 2014
(86) PCT No.: PCT/US14/54422

§ 371 Date: Mar. 4, 2016

Related U.S. Application Data
(60) Provisional application No. 61/873,944, filed on Sep. 5, 2013.

(51) Int. Cl. A61F 2/30; A61F 2/32; A61F 2/40; A61F 2/34

(52) U.S. Cl. CPC A61F 2/30767; A61F 2/34; A61F 2/30; A61F 2/32; A61F 2/40; A61F 2220/0008; A61F 2240/002

(57) ABSTRACT

Embodiments of the invention include surgical instruments and methods for providing a joint arthroplasty system configured for use with skeletal structures that may fee deformed or may have defects. In some embodiments, patient-matched surfaces arc prepared to engage with deformations or defects, and other reference points or surfaces are provided to give alignment guidance to enable appropriate placement of a reconstructive system.

### CROSS REFERENCE TO RELATED APPLICATIONS

This application claims the benefit of U.S. Provisional Application No. 61/873,944 filed Sep. 5, 2013, the contents of which are incorporated herein by reference in their entirety.

### FIELD OF THE INVENTION

The present invention relates generally to the field of medical devices, and more particularly relates to implants and reconstructive systems for attachment on deformed or defective skeletal structures.

### BACKGROUND

It is known in the prior art to provide supplemental implants that serve to restore the shape of skeletal structures that have been deformed or that include a defect. Such an implant is sometimes called an “augment”. Examples of several augments are found in International Publication No. WO2010/033473A2 entitled APPARATUS AND METHOD FOR ADDRESSING FEMORAL ACETABULAR IMPINGEMENT, which claims priority to U.S. Provisional Patent Application No. 61/098,105 filed September 18, 2008, the contents of each of these applications incorporated herein by reference in their entirety.

It is a continuing challenge to align medical devices such as reconstructive implants relative to the natural physiology of a patient. Proper alignment is essential for optimal wear of many devices. A new generation of instruments for aligning and placing orthopedic implants has been established with the use of patient-matched instruments. Typically, manufacturers use imaging data about the bones and other tissue of specific patients to manufacture patient-matched instruments that are used to perform surgery on the respective specific patients. Many patient-matched instruments include one or more surfaces that match with anatomical locations on a patient to provide a desired placement of the patient-matched instruments relative to the patient. The patient-matched instruments may also have surfaces with a fixed relationship to a medical device. Consequently, placement of patient-matched instruments on certain anatomical surfaces may enable a preplanned, desirable placement of a medical device on a patient. When a skeletal structure is deformed or includes some type of defect, it may also be a challenge to place a medical device on a patient because (1) the patient's skeletal structure may not provide adequate support, and/or (2) there may be a lack of anatomical references needed to align a device.

Improved devices and methods may provide a system that includes implants, which augment deformed or defective skeletal structures. Improved systems may also or in the alternative include implants, which help align and support devices including reconstructive systems.

### SUMMARY

An embodiment of the invention is an implant configured to augment a deformed skeletal structure of a patient and provide for support and alignment of a reconstructive system that includes a body with a size and shape similar to at least a portion of the size and shape of a volume missing from the deformed skeletal structure. The body may include a patient-matched surface configured to couple with at least a portion of the deformed skeletal structure in a planned location, and a reconstructive system interface surface configured to couple with one or more components of a reconstructive system to support and align the reconstructive system.

Another embodiment of the invention is a joint arthroplasty system that includes a reconstructive system and an implant. The reconstructive system may include a first component and a second component, each of which is configured to couple to respective skeletal structures and to contact the other component to facilitate articulation between the respective skeletal structures of a patient. The implant may include an implant configured to couple with the reconstructive system and to augment a deformed skeletal structure of the patient. The implant may have a body with a size and shape similar to at least a portion of the size and shape of a volume missing from the deformed skeletal structure, a patient-matched surface configured to couple with at least a portion of the deformed skeletal structure in a planned location, and a reconstructive system interface surface configured to couple with one or more components of the reconstructive system to support and align the reconstructive system.

Yet another embodiment of the invention is a method of providing a joint arthroplasty system. The method may include receiving data regarding a skeletal structure with a defect, determining a planned size and orientation for a reconstructive system to be coupled to the skeletal structure with a defect, and designing an implant that will at least in part correct the defect and will provide one or more references for the placement of the reconstructive system relative to the skeletal structure. The method may also include manufacturing the implant with a patient-matched surface configured to couple with at least a portion of the skeletal structure with a defect and one or more references for the placement of the reconstructive system, and providing the implant in combination with or to be joined with the reconstructive system.

### BRIEF DESCRIPTION OF THE DRAWINGS

FIG. 1 is a perspective view of a portion of a pelvis.

FIG. 2 is a cross-sectional view of a portion of the pelvis of FIG. 1.

FIG. 3 is a cross-sectional view of a portion of a pelvis similar to the pelvis of FIG. 1, but where a portion of the pelvis has been deformed or includes a defect.

FIG. 4 is a perspective view of an implant and a portion of a reconstructive system in place on the pelvis of FIG. 3.

FIG. 5 is a cross-sectional view of the implant of FIG. 4, including additional portions of a reconstructive system.

FIG. 6 is another perspective view of the implant and the portion of the reconstructive system illustrated in FIG. 4.

FIG. 7 is a cross-sectional view of a portion of a pelvis similar to the pelvis of FIG. 1, but where a portion of the pelvis has been deformed or includes a defect.

FIG. 8 is a cross-sectional view of an implant and portions of a reconstructive system taken through the same section as FIG. 7.

FIG. 9 is a cross-sectional view of the implant of FIG. 8 with additional portions of the reconstructive system illustrated.

FIG. 10 is a plan view of the implant, reconstructive system, and a portion of the pelvis of FIG. 9.

FIG. 11 is a cross-sectional view of an implant and portions of a reconstructive system taken through the same section as FIG. 7.

FIG. 12 is a plan view of the implant, reconstructive system, and a portion of the pelvis of FIG. 11.

### DETAILED DESCRIPTION OF THE EMBODIMENTS

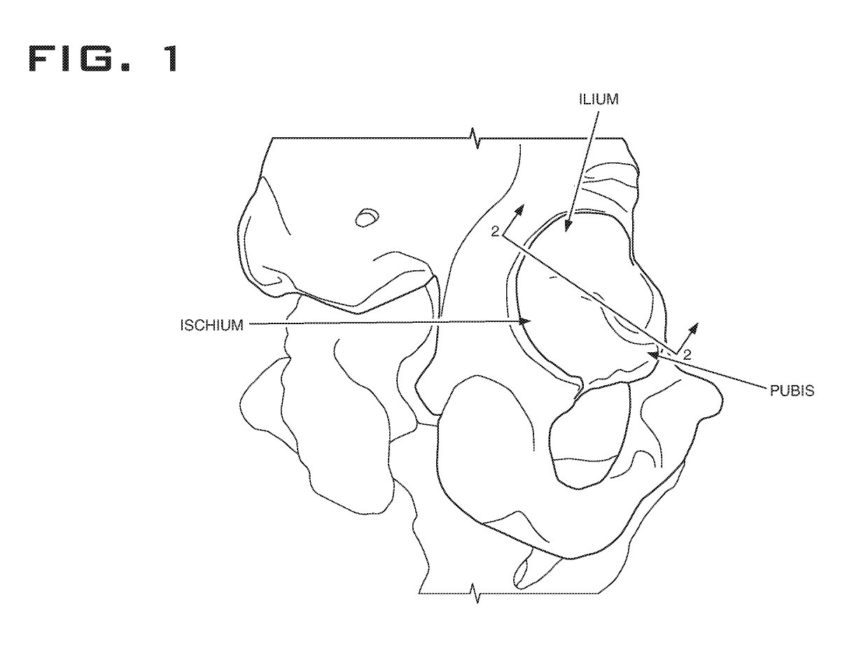

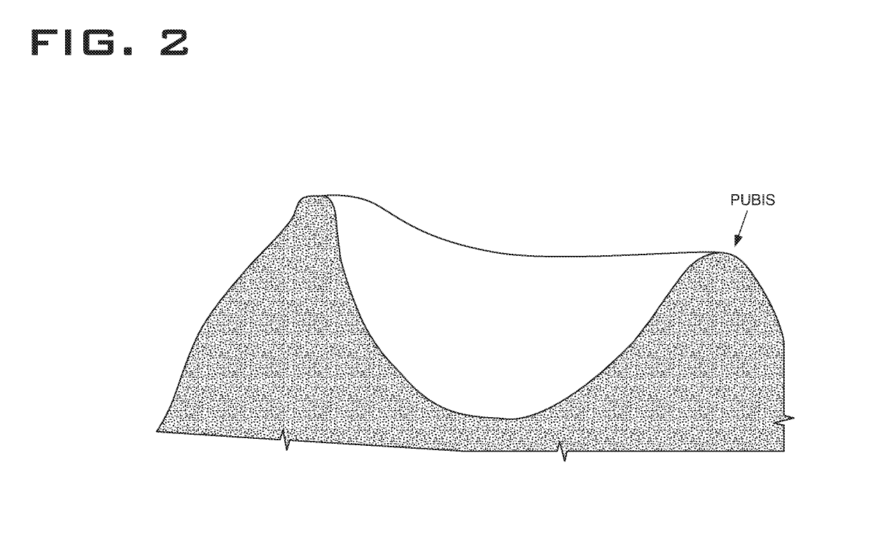

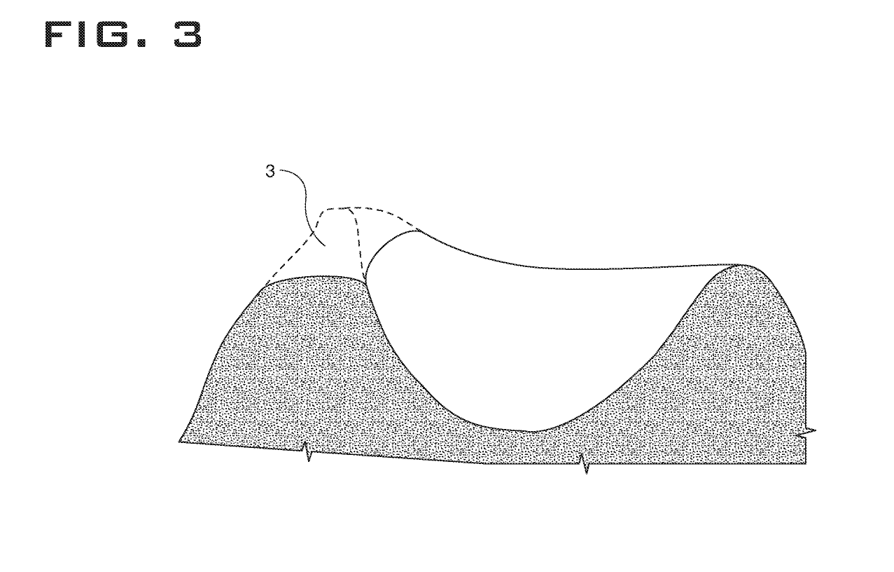

A perspective view of a portion of a typical pelvis is shown in FIG. 1. For the purpose of orienting the pelvis and identifying the parts of the acetabulum, the ischium, ilium, and pubis are designated in FIG. 1. A cross-sectional view of the pelvis of FIG. 1 is illustrated in FIG. 2. A cross-sectional view of a pelvis similar to the pelvis in FIG. 1, but where a portion of the pelvis has been deformed or includes a defect, is illustrated in FIG. 3. Such a deformity or defect may be caused by any type of wear, growth, disease, condition or trauma. For example and without limitation, a deformity or defect may be caused by impingement resulting from extra bone on the femoral neck repeatedly contacting the rim of the acetabulum. Similarly, a deformity or defect may be caused by impingement resulting from extra bone, such as a prominence, on the acetabular rim or other portion of the pelvis repeatedly being contacted by a femur or other tissue.

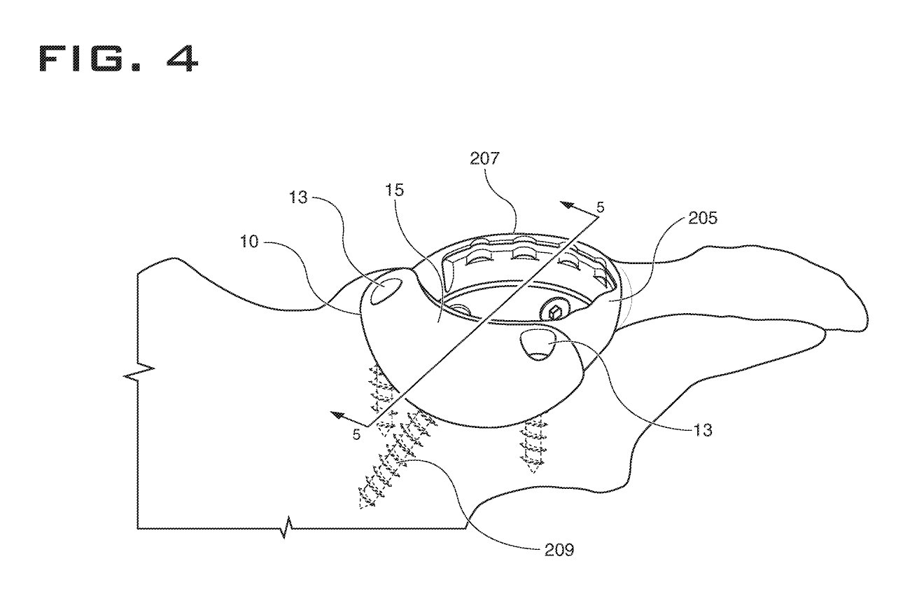

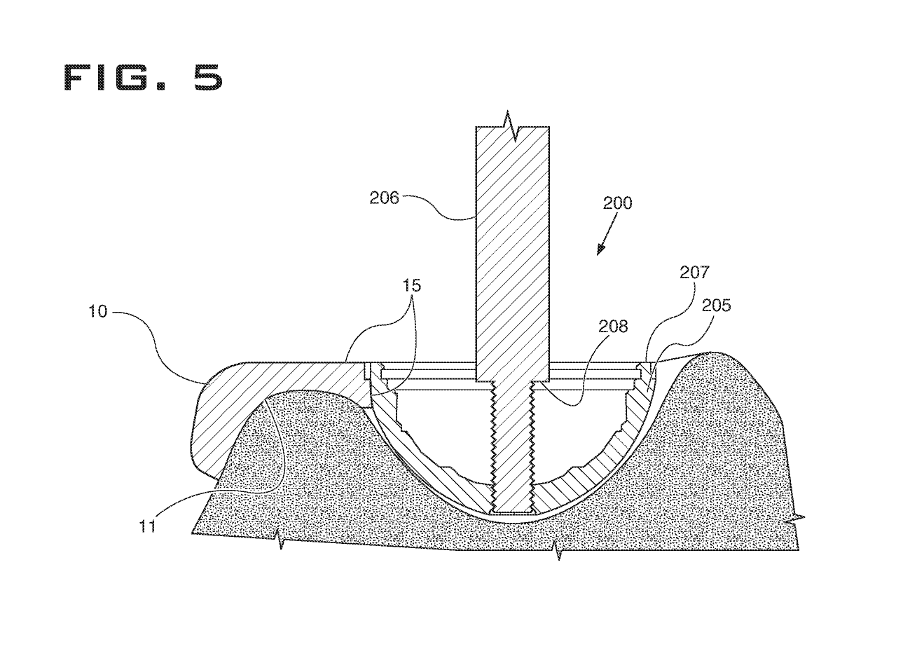

In an example embodiment illustrated in FIGS. 4-6, an implant 10 is configured to augment a deformed skeletal structure of a patient and provide support and alignment of a reconstructive system 200. In various embodiments, an augment may be used to provide support alone or alignment alone, or some embodiments of an augment may include the capacity to provide both support and alignment. A first component of the reconstructive system 200 shown in FIGS. 4-6 is an acetabular cup 205. An inserter 206 is shown in FIG. 5 that may be used to position, manipulate and impact the acetabular cup 205. The illustrated inserter 206 is coupled to the acetabular cup 205 by a threaded connection. Some embodiments may also include a spacer (not shown) between a shoulder 208 (FIG. 5) and the acetabular cup 205 to further stabilize the connection between the inserter 206 and the acetabular cup 205.

The body of the implant 10 illustrated in FIGS. 4-6 has a size and shape similar to a portion of the deformed portion of the pelvis illustrated in FIG. 3. Comparing FIG. 5 to FIG. 3, the body of the implant 10 shown in FIG. 5 in cross-section has a size and shape similar to the size and shape of a least a portion of a volume 3 (FIG. 3) missing from the deformed skeletal structure. In some embodiments, the size and shape of the body of the implant 10 may be substantially the same as the volume of a defect or deformation, but in other embodiments, an implant may be configured to provide additional features that, for example, help support or align a reconstructive system or another implant, system or device. The illustrated implant 10 includes fastener holes 13 (FIG. 4) configured to receive respective fasteners with which the body of the implant 10 may be coupled to the deformed skeletal structure. Fasteners of any effective type placed through any effective number or arrangement of holes may be employed in other embodiments.

As shown in FIG. 5, the body of the implant 10 includes a patient-matched surface 11 configured to couple with at least a portion of the deformed skeletal structure. In this embodiment, the patient-matched surface 11 is configured to couple with a deformed acetabulum. In other instances, a patient-matched surface may be configured to couple with another part of a hip bone or another bone adjacent to another joint, such as a shoulder bone. In various embodiments, coupling of the patient-matched surface 11 to at least a portion of the deformed skeletal structure may include alignment of some or all specific features of the patient-matched surface with the deformed skeletal structure or general matching of surface features. In the illustrated embodiment, the patient-matched surface 11 is configured to couple with the deformed skeletal structure in a planned location that has been evaluated by imaging the location and determining the size and shape of the planned location. The patient-matched surface 11 of the illustrated embodiment contacts and couples with both the deformed skeletal structure and skeletal structure that has not been deformed. As used here, the term couple may include one or more of matching in shape, matching in size, aligning with, mating, and being fixed to, including fixation with fasteners and adhesives. In some embodiments, a patient-matched surface may couple with only one of a deformed skeletal structure and skeletal structure that has not been deformed. A patient-matched surface of various embodiments may be generated by milling or casting processes, by any type of three-dimensional printing or deposition based processes, or by any other effective process.

The patient-matched surface 11 illustrated in FIG. 5 shows a surface that is configured to match with a portion of the deformed skeletal structure without the deformed skeletal structure being surgically reshaped. In this example, a deformity exists in a patient's body, as shown in FIG. 3. The patient-matched surface 11 is configured to match with a portion of the deformed skeletal structure without a surgeon reshaping the deformed skeletal structure to fit with or in preparation to receive the implant 10. In other embodiments, surgical reshaping may be necessary or desirable to fit a particular implant with a skeletal structure.

A reconstructive system interface surface 15, which is part of the body of the implant 10, is illustrated in FIGS. 4-6. The illustrated reconstructive system interface surface 15 is configured to couple with one or more components of a reconstructive system, such as the acetabular cup 205, to support and align the reconstructive system. As used here, the term couple may include one or more of matching in shape, matching in size, aligning with, mating, and being fixed to, including fixation with fasteners and adhesives. The reconstructive system interface surface 15, as illustrated in FIG. 5, includes portions that are not coplanar with one another and that are located generally on two sides of the body of the implant 10. One portion of the reconstructive system interface surface 15 conforms to the curved part of the acetabular cup 205, and another portion is configured to be coplanar with a top 207 of the acetabular cup 205 when the reconstructive system 200 is aligned as planned. A reconstructive system interface surface and a reconstructive system may be designated as aligned when the reconstructive system is in a planned relationship with an implant, and consequently the implant is in a planned relationship with a patient's anatomy. In some embodiments, a reconstructive system interface surface may be on a single side of an implant or on two or more sides of an implant, may include either or both planar and non-planar portions, and may or may not specifically conform to one of the shapes of a reconstructive system.

In another embodiment where an implant has at least two planar reconstructive system interface surfaces, a first planar portion of the reconstructive system interface surface may be configured to align with a first planar portion of the reconstructive system, and a second planar portion of the reconstructive system interface surface may be configured to align with a second planar portion of the reconstructive system when the reconstructive system is aligned as planned. In some such embodiments, the two planar reconstructive system interface surfaces are not coplanar.

In some embodiments, the reconstructive system interface surface is not a bearing surface between articulating portions of any combination of skeletal structures and components of a reconstructive system. In contrast, for example, a surface of the implant illustrated and described in International Publication No. WO2010/033473A2, the contents of which have been incorporated by reference in their entirety, is an interface between articulating portions of skeletal structures. The reconstructive system interface surface 15 illustrated in FIG. 5 is not a bearing surface between articulating portions of any combination of skeletal structures and components of a reconstructive system.

The embodiment illustrated in FIGS. 4-6 represents components of a joint arthroplasty system including the reconstructive system 200 and the implant 10 configured to couple with the reconstructive system 200 and to augment a deformed skeletal structure of a patient. In various embodiments, an augment may be used to provide support alone or alignment alone, or some embodiments of an augment may include the capacity to provide both support and alignment. A first component of the reconstructive system 200 shown is the acetabular cup 205. The acetabular cup 205 is configured to couple to the pelvis illustrated in FIGS. 4-6, and to contact a second component of the reconstructive system that is configured to be coupled to another skeletal structure of the patient to facilitate articulation between the skeletal structures. In some embodiments, the second component is a femoral component of a hip arthroplasty system. In other embodiments, the first and second components may be components of other joint arthroplasty systems, such as but not limited to, shoulder, knee, ankle, foot, elbow, wrist, and spine arthroplasty systems.

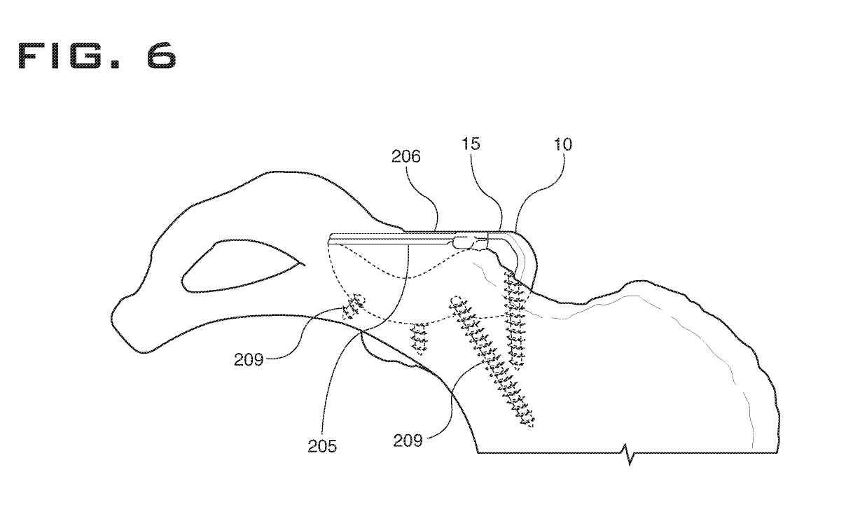

The inserter 206 shown in FIG. 5 may be used to position, manipulate and impact the acetabular cup 205. The inserter 206 illustrated is coupled to the acetabular cup 205 by a threaded connection. Some embodiments may also include a spacer (not illustrated) between a shoulder 208 (FIG. 5) and the acetabular cup 205 to further stabilize the connection between the inserter 206 and the acetabular cup 205. The embodiment shown in FIGS. 4 and 6 includes fasteners 209 for coupling the first component (acetabular cup 205) to the pelvis. Coupling for other embodiments may be accomplished by fasteners of other types, cements or adhesives of any type, or by any effective mechanism. Similarly, the second component may be coupled to a respective skeletal structure by fasteners, cements, or adhesives of any type, or by any effective mechanism.

The implant 10 of the joint arthroplasty system, including its body, patient-matched surface, and reconstructive system interface surface, and including various alternative embodiments, is essentially the same as the implant 10 described above.

The first component, as embodied in the acetabular cup 205, includes a planar surface, namely the top 207 of the acetabular cup 205, that is substantially coplanar with a planar area of the reconstructive system interface surface 15 of the implant 10 when the reconstructive system 200 is aligned as planned. A reconstructive system interface surface and a reconstructive system may be designated as aligned when the reconstructive system is in a planned relationship with an implant, and consequently the implant is in a planned relationship with a patient's anatomy.

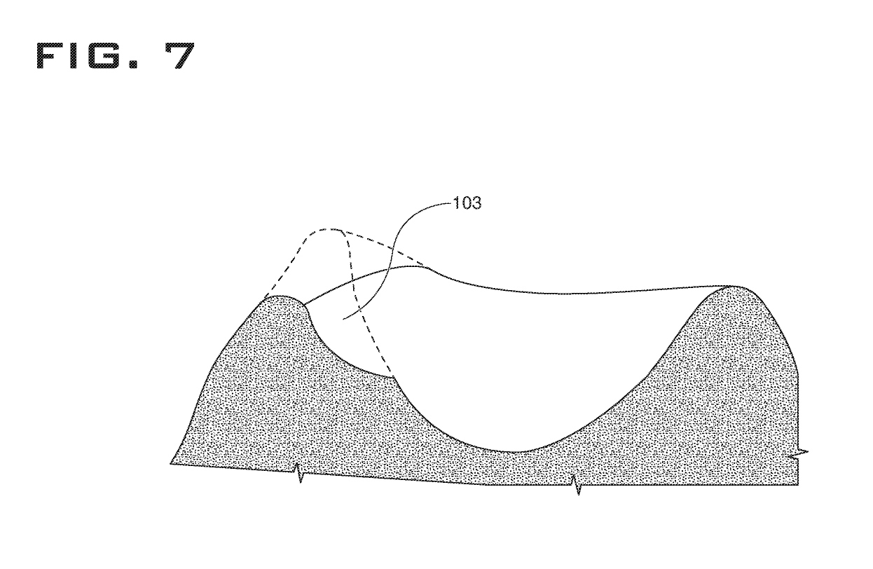

A cross-sectional view of a pelvis similar to the pelvis in FIG. 1, but where a portion of the pelvis has been deformed or includes a defect, is illustrated in FIG. 7. Such a deformity or defect may be caused by any type of wear, growth, disease, condition or trauma. For example and without limitation, a deformity or defect may be caused by impingement resulting from extra bone on the femoral neck repeatedly contacting the rim of the acetabulum. Similarly, a deformity or defect may be caused by impingement resulting from extra bone such as, for example, a prominence on the acetabular rim or other portion of the pelvis repeatedly being contacted by a femur or other tissue.

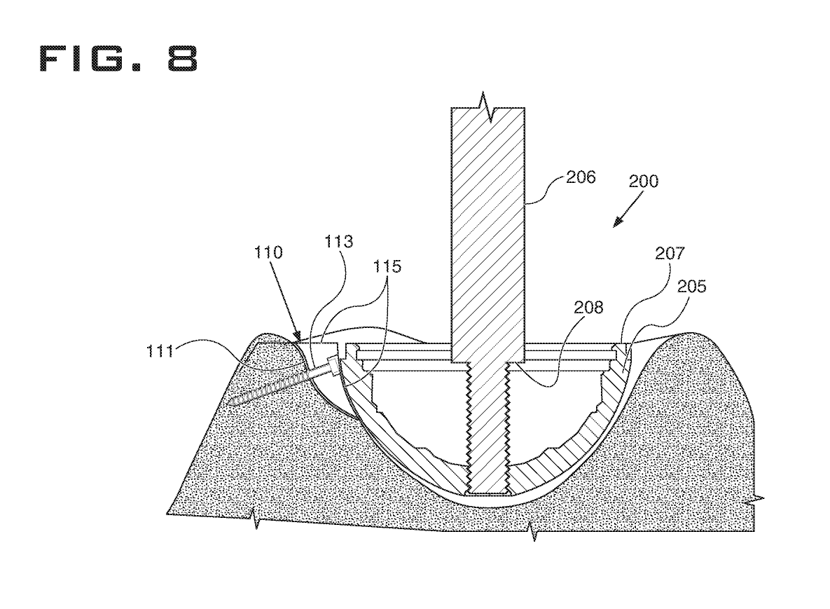

In an example embodiment and with regard to variations on the embodiments illustrated in FIGS. 8-12, an implant 110, 1110 is configured to augment a deformed skeletal structure of a patient and provide support and alignment of reconstructive system 200, 1200. An augment may be used to provide support alone or alignment alone, or some embodiments of an augment may include the capacity to provide both support and alignment. A first component of the reconstructive system 200, 1200 shown in FIGS. 8-12 is an acetabular cup 205, 1205. The inserter 206 shown in FIGS. 8, 9 and 11 may be used to position, manipulate, and impact the acetabular cup 205, 1205. The inserter 206 illustrated is coupled to the acetabular cup 205 by a threaded connection. Some embodiments may also include a spacer (not illustrated) between the shoulder 208 and the acetabular cup 205, 1205 to further stabilize the connection between the inserter 206 and the acetabular cup 205, 1205.

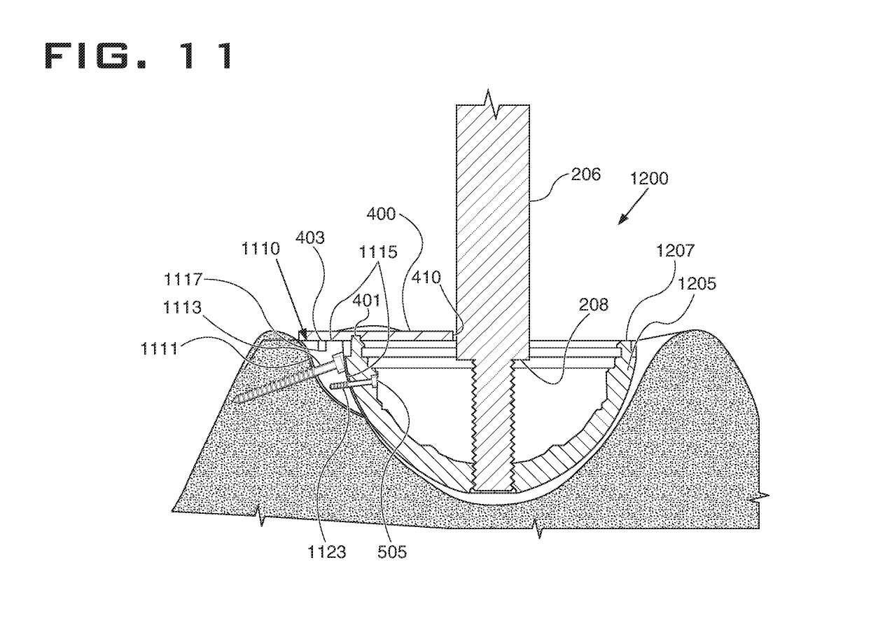

The body of the implant 110, 1110 illustrated in FIGS. 8-12 has a size and shape similar to a portion of the deformed portion of the pelvis illustrated in FIG. 7. Comparing FIGS. 8, 9 and 11 to FIG. 7, the body of the implant 110, 1110 shown in FIGS. 8, 9 and 11 in cross-section has a size and shape similar to the size and shape of a least a portion of a volume 103 (FIG. 7) missing from the deformed skeletal structure. In some embodiments, the size and shape of the body of the implant 110, 1110 may be substantially the same as the volume of a defect or deformation, but in other embodiments an implant may be configured to provide additional features that, for example, help support or align a reconstructive system or another implant, system or device. The implant 1110 shown in FIG. 11 includes an opening 1117 in the reconstructive system interface surface 1115 that is configured to receive a protrusion 403 of the reconstructive system 1200 when the reconstructive system 1200 is aligned. In other embodiments, a reconstructive system interface surface may include a protrusion that is configured to be inserted into an opening in the reconstructive system when the reconstructive system is aligned. Alternatively, any type of positive lock or stop between a reconstructive system and an implant at a planned location or position may be used to assist with alignment. The illustrated implant 110, 1110 includes fastener holes 113, 1113 (FIGS. 8, 9 and 11) configured to receive respective fasteners with which the body of the implant 110, 1110 may be coupled to the deformed skeletal structure. Fasteners of any effective type placed through any effective number or arrangement of holes may be employed in other embodiments. The implant 1110 shown in FIG. 11 includes a second fastener hole 1123 configured to receive a fastener with which the body of the implant 1110 may be coupled to one or more components of the reconstructive system. As shown in FIG. 11, a fastener 505 is employed to couple the acetabular cup 1205 to the body of the implant 1110. Any number of similar fasteners may be used and it is contemplated that any effective pattern of fasteners may be used. Such coupling may be accomplished with fasteners of other types, adhesives, or any effective mechanism in other embodiments. In various embodiments, an implant may be coupled with a reconstructive system before or after either or both are placed in contact a patient's skeletal structure. For example and without limitation, an implant may be secured alone first, and then a reconstructive system may be coupled to the implant, or the implant and reconstructive system may be coupled together and placed in contact with a patient's skeletal structure simultaneously. In the later example, a fastener between the implant and the skeletal structure may be used, or the implant may only be used for alignment purposes, and fasteners for the reconstructive system, such as fasteners 209 (FIGS. 4 and 6), may be used alone.

As may be seen in FIGS. 8, 9 and 11, the body of the implant 110, 1110 includes a patient-matched surface 111, 1111 configured to couple with at least a portion of the deformed skeletal structure. As illustrated, the patient-matched surface 111, 1111 is configured to couple with a deformed acetabulum. In other examples, a patient-matched surface may be configured to couple with another part of a hip bone or another bone adjacent to another joint, such as a shoulder bone. In various embodiments, coupling of the patient-matched surface 111, 1111 to at least a portion of the deformed skeletal structure may include alignment of some or all specific features of the patient-matched surface with the deformed skeletal structure or general matching of surface features. In the illustrated embodiment, the patient-matched surface 111, 1111 is configured to couple with the deformed skeletal structure in a planned location that has been evaluated by imaging the location and determining the size and shape of the planned location. As used here, the term couple may include one or more of matching in shape, matching in size, aligning with, mating, and being fixed to, including fixation with fasteners and adhesives. In some embodiments, a patient-matched surface may couple with only one of a deformed skeletal structure and skeletal structure that has not been deformed. A patient-matched surface of various embodiments may be generated by milling or casting processes, by any type of three-dimensional printing or deposition based processes, or by any other effective process.

The patient-matched surface 111, 1111 illustrated in FIGS. 8, 9 and 11 shows a surface that is configured to match with a portion of the deformed skeletal structure without the deformed skeletal structure being surgically reshaped. In this example, a deformity exists in a patient's body, as shown in FIG. 7. The patient-matched surface 111, 1111 is configured to match with a portion of the deformed skeletal structure without a surgeon reshaping the deformed skeletal structure to fit with or in preparation to receive the implant 110, 1110. In other embodiments, surgical reshaping may be necessary or desirable to fit a particular implant with a skeletal structure.

A reconstructive system interface surface 115, 1115, which is part of the body of the implant 110, 1110, is illustrated in FIGS. 8, 9 and 11. The illustrated reconstructive system interface surface 115, 1115 is configured to couple with one or more components of a reconstructive system such as, for example, the acetabular cup 205, 1205, to support and align the reconstructive system. As used here, the term couple may include one or more of matching in shape, matching in size, aligning with, mating, and being fixed to, including fixation with fasteners and adhesives. The reconstructive system interface surface 115, 1115, as illustrated in FIGS. 8, 9 and 11, includes portions that are not coplanar with one another and that are located generally on two sides of the body of the implant 110, 1110. One portion of the reconstructive system interface surface 115, 1115 conforms to the curved part of the acetabular cup 205, 1205, and another portion is configured to be coplanar with a top 207, 1207 of the acetabular cup 205, 1205 when the reconstructive system 200, 1200 is aligned as planned. A reconstructive system interface surface and a reconstructive system may be designated as aligned when the reconstructive system is in a planned relationship with an implant, and consequently the implant is positioned in a planned relationship with a patient's anatomy. In some embodiments, a reconstructive system interface surface may be located on a single side of an implant, or on two or more sides of an implant, may include either or both planar and non-planar portions, and may or may not specifically conform to one of the shapes of a reconstructive system.

In some embodiments, the reconstructive system interface surface is not a bearing surface between articulating portions of any combination of skeletal structures and components of a reconstructive system. In contrast, for example, a surface of the implant illustrated and described in International Publication No. WO2010/033473A2, the contents of which are incorporated by reference in their entirety, is an interface between articulating portions of skeletal structures. The reconstructive system interface surface 115, 1115 illustrated in FIGS. 8, 9 and 11 is not a bearing surface between articulating portions of any combination of skeletal structures and components of a reconstructive system.

The embodiments illustrated in FIGS. 8-12 represent components of a joint arthroplasty system including the reconstructive system 200, 1200 and the implant 110, 1110 configured to couple with the reconstructive system 200, 1200 and to augment a deformed skeletal structure of a patient. In various embodiments, an augment may be used to provide support alone or alignment alone, or some embodiments of an augment may include the capacity to provide both support and alignment. A first component of the reconstructive system 200, 1200 shown is the acetabular cup 205, 1205. The acetabular cup 205, 1205 is configured to couple to the pelvis illustrated in FIGS. 8-12, and to contact a second component of the reconstructive system that is configured to be coupled to another skeletal structure of the patient to facilitate articulation between the skeletal structures. In some embodiments, the second component is a femoral component of a hip arthroplasty system. In other embodiments, the first and second components may be components of other joint arthroplasty systems, such as but not limited to, shoulder, knee, ankle, foot, elbow, wrist, and spine arthroplasty systems.

The inserter 206 shown in FIGS. 8, 9 and 11 may be used to position, manipulate and impact the acetabular cup 205, 1205. The illustrated inserter 206 is coupled to the acetabular cup 205, 1205 by a threaded connection. Some embodiments may also include a spacer (not shown) positioned between a shoulder 208 and the acetabular cup 205, 1205 to further stabilize the connection between the inserter 206 and the acetabular cup 205, 1205. Coupling of the first component to the pelvis may be accomplished by fasteners of any effective type, cements or adhesives of any type, or by any effective mechanism. Similarly, the second component may be coupled to a respective skeletal structure by fasteners, cements, or adhesives of any type, or by any effective mechanism.

The implant 110, 1110 of the joint arthroplasty system, including its body, patient-matched surface, and reconstructive system interface surface, and including various alternative embodiments, is essentially the same as the implant 110, 1110 described above.

The first component, as embodied in the acetabular cup 205, 1205, includes a planar surface, namely the top 207, 1207 of the acetabular cup 205, 1205, that is substantially coplanar with a planar area of the reconstructive system interface surface 115, 1115 of the implant 110, 1110 when the reconstructive system 200, 1200 is aligned as planned. A reconstructive system interface surface and a reconstructive system may be designated as aligned when the reconstructive system is in a planned relationship with an implant, and consequently the implant is in a planned relationship with a patient's anatomy.

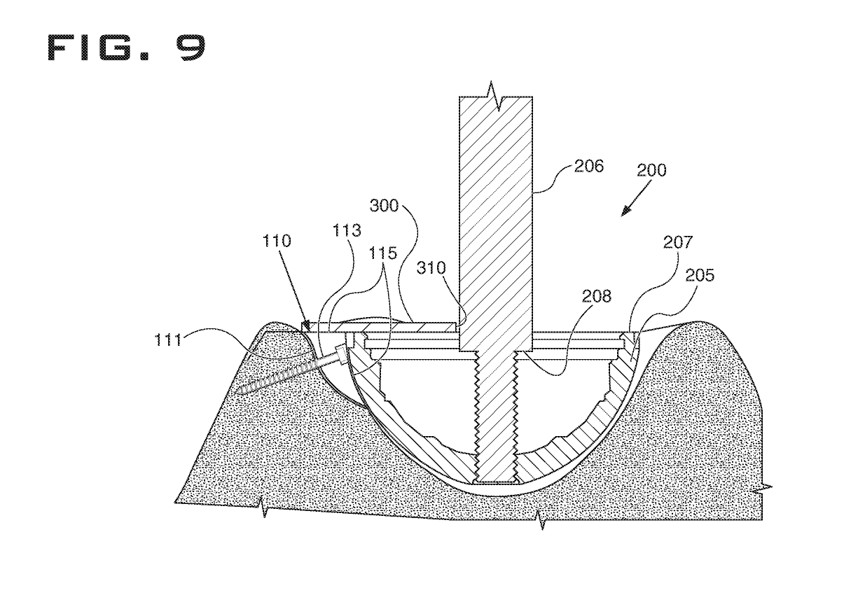

As shown in FIGS. 9-12, the joint arthroplasty system may include the reconstructive system 200, 1200 that includes an alignment guide 300, 400 configured to be coupled to the first component (acetabular cup 205, 1205) and to engage with the reconstructive system interface surface 115, 1115 when the reconstructive system 200, 1200 is aligned. As shown in FIG. 9, the alignment guide 300 may be engaged with the reconstructive system interface surface 115 by contact with the reconstructive system interface surface 115. As shown in FIG. 11, the alignment guide 400 may be engaged with the reconstructive system interface surface 1115 by contact with the reconstructive system interface surface 1115, and by insertion of the protrusions 403 into the openings 1117.

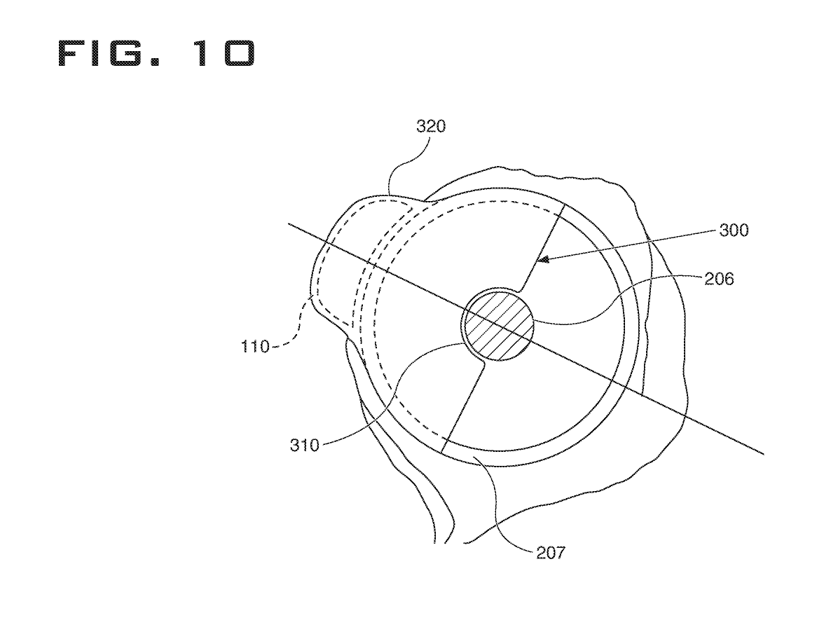

The alignment guide 300 illustrated in FIGS. 9-10 is joined to the reconstructive system 200 to provide alignment reference points for aligning the reconstructive system 200 with the implant 110, and consequently a patient's anatomy as planned. The alignment guide 300 may be joined to the reconstructive system 200 by any effective mechanism, and the joining may be fixed with fasteners or adhesives or may be held in place by a user. The alignment guide 300 includes a semicircular notch 310 and a tab 320. The semicircular notch 310 contacts the inserter 206 to fix the radial distance of the tab 320 from the inserter 206, and consequently the acetabular cup 205. The alignment guide 300 includes a lower surface configured to contact the top 207 of the acetabular cup 205 to establish a reference plane for alignment of the reconstructive system. With these references established, the tab 320 of the alignment guide 300 may be used as a guide relative to the implant 110.

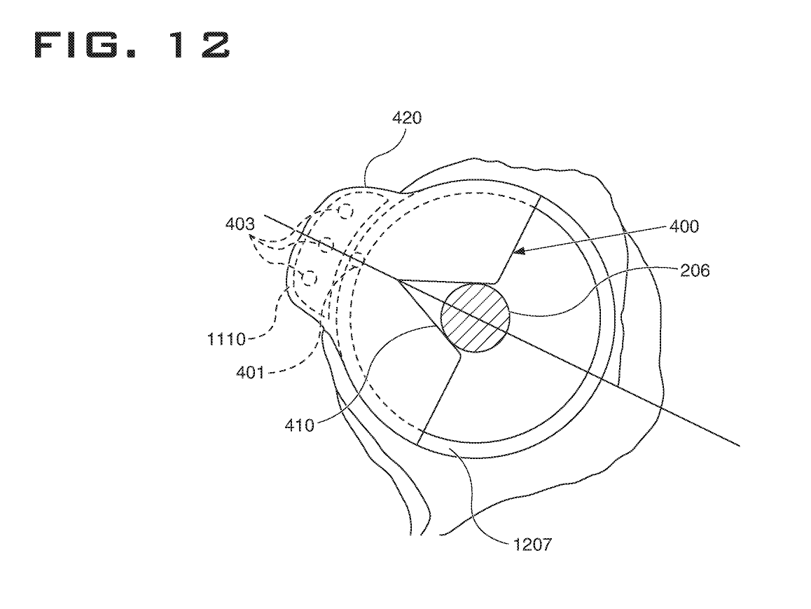

The alignment guide 400 illustrated in FIGS. 11 and 12 is joined to the reconstructive system 1200 to provide alignment reference points for aligning the reconstructive system 1200 with the implant 1110, and consequently a patient's anatomy as planned. In the illustrated embodiment, the alignment guide 400 also includes an opening 401 into which a protrusion extending from the top 1207 of the acetabular cup 1205 may be inserted to assist with fixing the alignment guide 400 relative to the acetabular cup 1205. The alignment guide 400 may be joined to the reconstructive system 1200 by any effective mechanism, and the joining may be fixed with fasteners or adhesives or may be held in place by a user. The alignment guide 400 includes a V-notch 410 and a tab 420. The V-notch 410 contacts the inserter 206 to fix the radial distance of the tab 420 from the inserter 206, and consequently the acetabular cup 1205. The alignment guide 400 includes a lower surface configured to contact the top 1207 of the acetabular cup 1205 to establish a reference plane for alignment of the reconstructive system. As noted above, the alignment guide 400 includes protrusions 403 that are configured to be inserted into openings 1117 in the reconstructive system interface surface 1115 when the reconstructive system 1200 is aligned with the implant 1110. With these references established, the tab 420 of the alignment guide 400 may be used as a guide relative to the implant 1110.

One embodiment of the invention includes a method of providing a joint arthroplasty system. Embodiments may include the act of receiving data regarding a skeletal structure with a defect. A defect may be caused by any type of wear, growth, disease, condition or trauma, or may be a deformity. The data received may be, for example, size and shape characteristics obtained with an imaging device. MRI scans or CT scans may be used to automatically obtain three-dimensional models of a patient's anatomy. Alternatively, two-dimensional imaging devices, such as a radiograph, may be used from more than one angle to approximate a three-dimensional model that may provide adequate size and shape characteristics. Any other type of imaging device capable of determining size and shape characteristics of patients is also contemplated. The act of receiving data regarding a skeletal structure with a defect may also include receiving data identifying the defect. For example and without limitation, the act of receiving data may include receiving data identifying a defect, such as the defect illustrated in FIG. 3, by the missing volume 3 in the skeletal structure. The act of receiving data regarding a skeletal structure with a defect may also include receiving data identifying a pelvic plane for reference. Identification of a pelvic plane may be useful in orienting a reconstructive system for effective operation.

Method embodiments may also include determining a planned size and orientation for a reconstructive system to be coupled to the skeletal structure with a defect. Specific examples illustrated in the figures include determining a planned size and orientation for the implant 10 (FIGS. 4-6), the implant 110 (FIGS. 8-10), and the implant 1110 (FIGS. 11 and 12). The act of determining a planned size and orientation for the reconstructive system may also include virtually orienting and acetabular cup 205, 1205 relative to a pelvic plane. The act of virtually orienting an acetabular cup 205, 1205 may include orienting the cup for optimal support and freedom of motion of components of the reconstructive system. For example, when a femoral stem is a part of the illustrated reconstructive system 200, 1200, then the virtual orientation of the acetabular cup 205, 1205 may be accomplished to optimize support and freedom of motion of the femoral stem relative to an acetabular cup 205, 1205. An example virtual orientation of the acetabular cup 205, 1205 may include orienting the cup with approximately 40 degrees inclination and approximately 20 degrees anteverted.

Method embodiments may also include the act of designing an implant that will at least in part correct the defect, and will provide one or more references for the placement of the reconstructive system relative to the skeletal structure. For example and without limitation, the implant 10, 110, 1110 (FIGS. 4-6 and 8-12) at least in part fills the defects identified by the volume 3, 103 (FIGS. 3 and 7). The correction of a defect may also include covering, reinforcing, or otherwise making the defect more hospitable to receiving an implant.

Some method embodiments include the act of manufacturing the implant with a patient-matched surface configured to couple with at least a portion of the skeletal structure having a defect. The act of manufacturing may also include producing an implant with one or more references for the placement of the reconstructive system. Embodiments disclosed herein of the implant 10, 110, 1110 provide nonlimiting examples of an implant manufactured with a patient-matched surface configured to couple with at least a portion of the skeletal structure with a defect. Similarly, the implant 10, 110, 1110 includes a reconstructive system interface surface 15, 115, 1115 that provides one or more references for the placement of the reconstructive system 200, 1200. References for placement may include but are not necessarily limited to the surfaces, openings, protrusions, and other elements disclosed herein in association with the implant 10, 110, 1110.

Some method embodiments may also include providing the implant 10, 110, 1110 in combination with or to be joined with the reconstructive system 200, 1200. Implants and reconstructive systems may be provided separately or together. In some embodiments, implants and reconstructive systems may be provided in a kit. Additionally, implants and reconstructive systems may be combined by a manufacturer, or may be combined by a party who manufactures or creates all, part, or none of the components or instructions.

Various embodiments of a system wholly or its components individually may be made from any biocompatible material. For example and without limitation, biocompatible materials may include in whole or in part: non-reinforced polymers, reinforced polymers, metals, ceramics, adhesives, reinforced adhesives, and combinations of these materials. Reinforcing of polymers may be accomplished with carbon, metal, or glass or any other effective material. Examples of biocompatible polymer materials include polyamide base resins, polyethylene, low density polyethylene, polymethylmethacrylate (PMMA), polyetheretherketone (PEEK), polyetherketoneketone (PEKK), a polymeric hydroxyethylmethacrylate (PHEMA), and polyurethane, any of which may be reinforced. Example biocompatible metals include stainless steel and other steel alloys, cobalt chrome alloys, tantalum, titanium, titanium alloys, titanium-nickel alloys such as Nitinol and other superelastic or shape-memory metal alloys.

Terms such as side, top, and the like have been used relatively herein. However, such terms are not limited to specific coordinate orientations, but are used to describe relative positions referencing particular embodiments. Such terms are not generally limiting to the scope of the claims made herein. Any embodiment or feature of any section, portion, or any other component shown or particularly described in relation to various embodiments of similar sections, portions, or components herein may be interchangeably applied to any other similar embodiment or feature shown or described herein.

While embodiments of the invention have been illustrated and described in detail in the disclosure, the disclosure is to be considered as illustrative and not restrictive in character. All changes and modifications that come within the spirit of the invention are to be considered within the scope of the disclosure.

One form of the invention is directed to an implant configured to augment a deformed skeletal structure of a patient and provide for support and alignment of a reconstructive system, with the implant including a body with a size and shape similar to at least a portion of the size and shape of a volume missing from the deformed skeletal structure, the body including a patient-matched surface configured to couple with at least a portion of the deformed skeletal structure in a planned location; and a reconstructive system interface surface configured to couple with one or more components of a reconstructive system to support and align the reconstructive system.

In one aspect of the invention, the patient-matched surface is configured to match with a portion of the deformed skeletal structure without the deformed skeletal structure being surgically reshaped.

In another aspect of the invention, the patient-matched surface is configured to couple with a deformed hip bone.

In another aspect of the invention, the patient-matched surface is configured to couple with a deformed acetabulum.

In another aspect of the invention, the patient-matched surface is configured to couple with a deformed shoulder bone.

In another aspect of the invention, the reconstructive system interface surface includes a planar surface that is configured to be substantially coplanar with a surface of the reconstructive system when the reconstructive system is aligned.

In another aspect of the invention, the reconstructive system interface surface includes a protrusion that is configured to be inserted into an opening in the reconstructive system when the reconstructive system is aligned.

In another aspect of the invention, the reconstructive system interface surface includes an opening that is configured to receive a protrusion of the reconstructive system when the reconstructive system is aligned.

In another aspect of the invention, the reconstructive system interface surface is not a bearing surface between articulating portions of any combination of skeletal structures and components of a reconstructive system.

In another aspect of the invention, the body includes a first fastener hole configured to receive a fastener with which the body may be coupled to the deformed skeletal structure.

In another aspect of the invention, the body includes a second fastener hole configured to receive a fastener with which the body may be coupled to one or more components of the reconstructive system.

Another form of the invention is directed to a joint arthroplasty system comprising a reconstructive system including a first component and a second component and with each configured to couple to respective skeletal structures and to contact the other component to facilitate articulation between the respective skeletal structures of a patient; and an implant configured to couple with the reconstructive system and to augment a deformed skeletal structure of the patient, wherein the implant has a body with a size and shape similar to at least a portion of the size and shape of a volume missing from the deformed skeletal structure, and with the body including a patient-matched surface configured to couple with at least a portion of the deformed skeletal structure in a planned location; and a reconstructive system interface surface configured to couple with one or more components of the reconstructive system to support and align the reconstructive system.

In one aspect of the invention, the first component is an acetabular component and the second component is a femoral component of a total hip arthroplasty system.

In another aspect of the invention, the reconstructive system includes one or more fasteners for coupling one or both of the first component and the second component to respective separate skeletal structures.

In another aspect of the invention, the first component includes a planar surface that is substantially coplanar with a planar surface of the implant when the reconstructive system is aligned.

In another aspect of the invention, the reconstructive system includes an alignment guide configured to be coupled to the first component and to engage with the reconstructive system interface surface when the reconstructive system is aligned.

In another aspect of the invention, the alignment guide includes an opening that is configured to receive a protrusion of the implant when the reconstructive system is aligned.

In another aspect of the invention, the alignment guide includes a protrusion that is configured to be inserted into an opening in the implant when the reconstructive system is aligned.

In another aspect of the invention, the patient-matched surface is configured to match with a portion of the deformed skeletal structure without the deformed skeletal structure being surgically reshaped.

In another aspect of the invention, the patient-matched surface is configured to couple with a deformed hip bone.

In another aspect of the invention, the patient-matched surface is configured to couple with a deformed acetabulum.

In another aspect of the invention, the patient-matched surface is configured to couple with a deformed shoulder bone.

In another aspect of the invention, the reconstructive system interface surface includes a protrusion that is configured to be inserted into an opening in the reconstructive system when the reconstructive system is aligned.

In another aspect of the invention, the reconstructive system interface surface includes an opening that is configured to receive a protrusion of the reconstructive system when the reconstructive system is aligned.

In another aspect of the invention, the reconstructive system interface surface is not a bearing surface between articulating portions of any combination of skeletal structures and components of a reconstructive system.

In another aspect of the invention, the body includes a first fastener hole configured to receive a fastener with which the body may be coupled to the deformed skeletal structure.

In another aspect of the invention, the body includes a second fastener hole configured to receive a fastener with which the body may be coupled to one or more components of the reconstructive system.

In another aspect of the invention, the joint arthroplasty system includes one or more fasteners for one or more of coupling the body to the deformed skeletal structure and coupling the body to one or more components of the reconstruction system.

Another form of the invention is directed to a method of providing a joint arthroplasty system including receiving data regarding a skeletal structure with a defect; determining a planned size and orientation for a reconstructive system to be coupled to the skeletal structure with a defect; designing an implant that will at least in part correct the defect and will provide one or more references for the placement of the reconstructive system relative to the skeletal structure; manufacturing the implant with a patient-matched surface configured to couple with at least a portion of the skeletal structure with a defect and one or more references for the placement of the reconstructive system; and providing the implant in combination with or to be joined with the reconstructive system.

In one aspect of the invention, the act of receiving data regarding a skeletal structure with a defect includes receiving data identifying the defect.

In another aspect of the invention, the act of receiving data regarding a skeletal structure with a defect includes receiving data identifying a pelvic plane for reference.

In another aspect of the invention, the act of determining a planned size and orientation for the reconstructive system includes virtually orienting an acetabular cup relative to a pelvic plane.

In another aspect of the invention, the act of virtually orienting an acetabular cup includes orienting the cup for optimal support and freedom of motion of the components of the reconstructive system.

In another aspect of the invention, the act of orienting the cup for optimal support and freedom of motion of the components of the reconstructive system includes orienting the cup with approximately 40 degrees inclination and approximately 20 degrees anteverted.

In another aspect of the invention, act of manufacturing the implant with one or more references for the placement of the reconstructive system includes manufacturing a protrusion that is configured to be inserted into an opening in the reconstructive system when the reconstructive system is aligned.

In another aspect of the invention, the act of manufacturing the implant with one or more references for the placement of the reconstructive system includes manufacturing an opening that is configured to receive a protrusion of the reconstructive system when the reconstructive system is aligned.

In another aspect of the invention, the act of manufacturing the implant with one or more references for the placement of the reconstructive system includes manufacturing a fastener hole in the implant.

1. An implant configured to augment a deformed skeletal Structure of a patient and provide for support and alignment of a reconstructive-system, comprising:
a body with a size and shape similar to at least a portion of the size and shape of a volume missing from the deformed skeletal structure, the body comprising;
a patient-matched surface configured to couple with, at least a portion of the deformed skeletal structure in a planned location; and
a reconstructive system interface-surface configured to couple with one or more components of a reconstructive system to support and align the reconstructive system,

2. The implant of claim 1 wherein the patient-matched surface is configured to match with a portion of the deformed skeletal structure without the deformed skeletal structure being surgically reshaped,

3. The implant of claim 1 wherein the -patient-matched, surface is configured to couple with a deformed hip bone,.

4. The implant of claim 1 wherein the patient-matched surface is configured to couple with, a deformed acetabulum.

5. The implant of claim 1 wherein the patient-matched surface is configured to couple with a deformed shoulder bone,

6. The implant of claim 1 wherein the reconstructive system interface surface includes a planar surface that is configured to foe substantially coplanar with a surface of the reconstructive system when the reconstructive system is aligned,

7. The implant of claim 1 wherein the reconstructive system interface surface includes a protrusion that is configured to be inserted into an opening in the reconstructive system when the reconstructive system is aligned.

8. The implant, of claim 1 wherein the reconstructive system interface surface includes an opening that is configured to receive a protrusion of the reconstructive system when the reconstructive system is aligned,

9.The implant of claim 1 wherein the reconstructive system interface surface is not a bearing surface between articulating portions of any combination of skeletal structures and components of a reconstructive system.

10. The implant of-claim 1 wherein the body includes a first fastener hole configured to receives fastener with which the body may be coupled to the deformed skeletal structure,

11. The implant of claim 1 wherein the body includes a second fastener hole configured to receive a fastener with which the body may be coupled to one or more components of the reconstructive system,

12. A joint arthroplasty system, comprising:
a reconstructive system including a first component and a second component, each of which is configured to couple-to respective skeletal structures and to contact the other component to facilitate articulation between the respective skeletal structures of a patient; and
an implant configured to couple with the reconstructive system and to augment a deformed skeletal structure of the patient, wherein the implant has a body with a size and shape similar -to at least a portion of the size and shape of a volume missing from the deformed skeletal structure, the body comprising:
a patient-matched surface configured to couple with at least a portion of the deformed skeletal structure in a planned location; and
a reconstructive system interface surface configured to couple with one or more components of the reconstructive system to support and align the reconstructive system.

13. The joint arthroplasty system of-claim 12 wherein the first component is an acetabular component and the second component is a femoral component of a total hip arthroplasty system.

14. The joint arthroplasty system of claim 12 wherein the reconstructive system includes one or more fasteners for coupling one or both, of the first component and the second component to respective separate skeletal structures..

15. The joint arthroplasty system of claim 12 wherein first component includes a planar surface that is substantially coplanar with a planar surface of the implant when the reconstructive system is aligned.

16. The joint arthroplasty system of claim 12 wherein the reconstructive system includes an alignment guide configured to he coupled to the first component and to engage with the reconstructive system interlace surface when the reconstructive system is aligned.

17. The joint arthroplasty system of claim 16 wherein the alignment guide includes an opening that is configured to receive a protrusion of the implant when the reconstructive system is aligned,

18. The joint arthroplasty system of claim 16 wherein the alignment guide includes a protrusion that is configured to be inserted into an opening in the implant when the reconstructive system is aligned.

19. The joint arthroplasty system of claim 12 wherein the patient-matched surface is configured to match with a portion of the deformed skeletal structure without the deformed skeletal structure being surgically reshaped.

20. The joint arthroplasty system, of claim 12 wherein the patient-matched surface is configured to couple with a deformed hip bone.

21. The joint arthroplasty system of claim 12 wherein the patient-matched surface is configured to couple with a deformed acetabulum.

22. The joint arthroplasty system of claim 12 wherein the patient-matched surface is configured to couple with a deformed shoulder bone.

23. The joint arthroplasty system of claim 12 wherein the reconstructive system interface surface includes a protrusion that is configured to be inserted into an opening in the reconstructive system when the reconstructive system is aligned,

24. The joint arthroplasty system of claim 12 wherein the reconstructive system interface surface includes an opening that is configured to receive a protrusion of the reconstructive system when the reconstructive system is aligned,

25. The joint arthroplasty system of claim 12 wherein the reconstructive system interface surface is not a bearing surface between articulating portions of any combination of skeletal structures and components of a reconstructive system,

26. The joint arthroplasty system of claim 12 wherein the body includes a first fastener hole configured to receive a fastener with which the body may be coupled to the deformed skeletal structure.

27. The joint arthroplasty system of claim 12 wherein the body includes a a second fastener hole configured to receive a fastener with which the body may be coupled to one or more components of the reconstructive system.

28. The joint arthroplasty system of claim 12 wherein the joint arthroplasty system includes one or more fasteners for one or more of coupling the body to the deformed skeletal structure and coupling the body to one or more components of the reconstruction system.

29. A method of providing a joint arthroplasty system, comprising: receiving data regarding a skeletal structure with a defect;
determining a planned size and orientation for a reconstructive system to be coupled to the skeletal structure with a defect;
designing an implant that will at least in part correct the defect and will provide one or more references for the placement of the reconstructive system relative to the skeletal structure;
manufacturing the implant with a patient-matched surface configured to couple with at least a portion of the skeletal structure-with a defect and one or more references for the placement of the reconstructive system; and
providing the implant in combination with or to be joined with the reconstructive system.

30.-37. (canceled)
